(19) United States
(12) Patent Application Publication
(10) Pub. No.: US 2026/0151222 A1
(43) Pub. Date: Jun. 4, 2026

(54) METHOD AND ASSEMBLY FOR PRODUCING AN INTRAOCULAR LENS

(71) Applicant: Carl Zeiss Meditec AG, Jena (DE)

(72) Inventors: Michael Thaller, Oberkochen (DE); Benjamin Schreiber, Oberkochen (DE); Thorben Badur, Oberkochen (DE); Francesca Nicoli, Oberkochen (DE); Jennifer-Magdalena Masch, Oberkochen (DE)

(21) Appl. No.: 19/458,483

(22) Filed: Jan. 23, 2026

Related U.S. Application Data
(63) Continuation of application No. PCT/EP2024/071100, filed on Jul. 25, 2024.

(30) Foreign Application Priority Data
Jul. 26, 2023 (DE) 10 2023 207 114.2

(51) Int. Cl. A61F 2/16

(52) U.S. Cl. CPC A61F 2/16; A61F 2240/001

(57) ABSTRACT

A method is for making an intraocular lens and includes providing a container which is transparent to electromagnetic radiation; providing a liquid, which can be cured via electromagnetic radiation, in the container; generating and/or providing a data set of images of an intraocular lens with at least one cavity, wherein the images contain projections of the intraocular lens with the at least one cavity from different directions; tomographically printing the curable liquid via electromagnetic radiation on the basis of the generated and/or provided data set in order to produce the intraocular lens with the at least one cavity; and deactivating the ability to cure the curable liquid in the at least one cavity. An assembly is for making an intraocular lens.

### CROSS-REFERENCE TO RELATED APPLICATIONS

This application is a continuation application of international patent application PCT/EP2024/071100, filed Jul. 25, 2024, designating the United States and claiming priority from German application 10 2023 207 114.2, filed Jul. 26, 2023, and the entire content of both applications is incorporated herein by reference.

### BACKGROUND

Intraocular lenses are conventionally produced by turning. For this purpose, the material of the intraocular lens is first produced by polymerization. Then blanks are cut out of the material. In the case of hydrophilic intraocular lenses, the blanks are secured to a turning machine, in particular via wax; in the case of hydrophobic intraocular lenses, the blanks are frozen on, in particular at about −20° C. A computer-controlled robotic arm equipped with a diamond tip machines the intraocular lens from the blank rotating in the turning machine. However, this is a complex and costly method. It is additionally disadvantageous that a diamond tip used in the turning operation leaves grooves on the surface of the intraocular lens, and the grooves impair an optical quality of the intraocular lens.

DE 10 2020 108 375 B3 also discloses a method of producing an intraocular lens by way of tomographic printing. The method comprises the steps of: providing a vessel which is transparent to electromagnetic radiation and in which there is a liquid that is curable by the electromagnetic radiation; irradiating the liquid with a set of images formed by the electromagnetic radiation, which each depict an intraocular lens, by radiation of each of the images of the set into the liquid at a different angle of incidence with respect to a reference plane that extends through the liquid, as a result of which the liquid is cured and the cured liquid forms the intraocular lens, with positioning of an actuator, a solar module and/or a sensor in the liquid and formation of the intraocular lens around the actuator, the solar module and/or the sensor.

Another known method is to fill a cavity in intraocular lenses with a liquid. This allows varying and adjustment of the optical properties of the intraocular lens.

### SUMMARY

It is an object of the disclosure to improve a method and assembly for producing an intraocular lens.

The object is, for example, achieved in accordance with the disclosure by a method according to various embodiments of the disclosure and an assembly according to various embodiments of the disclosure.

One of the basic concepts of the disclosure is to fill a cavity in the intraocular lens with the liquid used in tomographic printing, but to deactivate its ability to cure after the tomographic printing. In this way, the liquid remaining in the cavity can in particular no longer be cured by electromagnetic radiation. In particular, this can prevent further unwanted polymerization, such that the liquid remains in a liquid state thereafter. In particular, an active group of the monomer (for example, acrylate) is converted here to an inert group. This is based on the concept that using the technique of tomographic printing allows printing of the intraocular lens in a (mechanically) stabilizing solution which is provided by the curable liquid. If the tomographic printing produces (at least) one cavity in the intraocular lens, the curable liquid remains in the (at least) one cavity after the printing process has ended, since activating of the curable liquid for polymerization has not taken place therein, but the curable liquid can no longer leave the cavity since the liquid is trapped by the walls of the (at least one) cavity. In order to prevent subsequent unwanted curing or further polymerization, the curable liquid remaining in the (at least one) cavity is deactivated and the liquid state is maintained.

In particular, a method of producing an intraocular lens is provided, includes: providing a vessel transparent to electromagnetic radiation, providing a liquid curable by electromagnetic radiation in the vessel, creating and/or providing a dataset of images of an intraocular lens with at least one cavity, the images including projections of the intraocular lens with the at least one cavity from different directions, tomographically printing the curable liquid via electromagnetic radiation proceeding from the dataset created and/or provided to form the intraocular lens with the at least one cavity, and deactivating the curability of the curable liquid in the at least one cavity.

Also provided is an assembly for producing an intraocular lens, including a vessel transparent to electromagnetic radiation; a data processing device set up to create and/or provide a dataset of images of an intraocular lens with at least one cavity, the images including projections of the intraocular lens with the at least one cavity from different directions; a tomographic printing device set up for tomographic printing of a curable liquid provided in the vessel via electromagnetic radiation proceeding from the dataset created and/or provided to form the intraocular lens with the at least one cavity; and at least one deactivating means set up to deactivate the curability of the curable liquid in the at least one cavity or to prepare for and/or assist the deactivating operation.

One advantage of the method and the assembly is that a cavity in the intraocular lens no longer has to be separately filled with a liquid. This can avoid the problem of leakage that always exists in a subsequent filling operation, since access to the cavity and subsequent reclosure are no longer necessary.

The electromagnetic radiation is in the optical wavelength range in particular, especially in the visible and/or UV wavelength range. In particular, the curable liquid may have the properties which are described in DE 10 2020 108 375 B3. In particular, tomographic printing is implemented, in principle, in the manner described in DE 10 2020 108 375 B3. For creation and/or provision purposes, the dataset images can be calculated, for example, from a three-dimensional dataset (for example, CAD data) that includes the shape of the intraocular lens. In particular, this is an inverse process as used in tomographic imaging. For example, tomographic imaging is used in computed tomography. For example, tomographic imaging can make use of Radon transform. In particular, tomographic printing makes use of this inverse process in order, via the calculated images, to locally cure the curable liquid in a manner dependent on the light patterns in the dataset images.

In particular, the curable liquid contains a solution including a dissolved monomer and a photoinitiator that can trigger polymerization of the monomer in a radiation-dependent manner. Moreover, further substances may also be part of the curable liquid, for example fillers, optically excitable dyes or nanoparticles and/or medical actives. In particular, the curable liquid has a defined minimum viscosity of at least 100 mPa*s (cps). The minimum viscosity ensures in particular that the intraocular lens remains in the same position during tomographic printing. Alternatively or additionally, a support device may be provided to hold the intraocular lens in position in the course of tomographic printing.

Starting materials used for the curable liquid may in particular be hydroxyethyl methacrylate (HEMA), methyl methacrylate (MMA) or 2-ethoxyethyl methacrylate (EOEMA).

It may be the case that the intraocular lens is an accommodating intraocular lens. Moreover, the intraocular lens may additionally include an actuator, a solar module and/or a sensor that can be inserted into the curable liquid, allowing the intraocular lens to be printed around these articles. Moreover, the intraocular lens may include an optics element and at least one tactile element.

It may be the case that the intraocular lens produced by the method is subsequently processed further, for example by turning, mechanical polishing, laser polishing and/or laser cutting, et cetera.

In one embodiment, the deactivating includes impregnating and/or extracting the printed intraocular lens with and/or in a defined extraction liquid or a defined extraction gas. The terms “extraction liquid” and “extraction gas” are chosen here in particular solely for conceptual differentiation and refer in particular to a liquid or a gas that is used in the course of deactivating. In particular, the deactivating is brought about and/or supported in that the liquid or the gas removes (extracts) at least one constituent from the curable liquid remaining in the at least one cavity. For this purpose, the remaining curable liquid is removed from the vessel after the tomographic printing and the defined extraction liquid or the defined extraction gas is introduced into the vessel. The extraction liquid or the extraction gas leads to deactivating of the curable liquid or at least assists the deactivating.

In one embodiment, the curable liquid includes a monomer that is insoluble with respect to the defined extraction liquid and a photoinitiator that is soluble with respect to the defined extraction liquid, where the photoinitiator present in the at least one cavity is removed from the at least one cavity by the impregnating and/or extracting operation. This allows the photoinitiator present in the at least one cavity to be removed therefrom, such that it is no longer possible to activate the polymerization in the at least one cavity. For example, it may be the case that the curable liquid includes hydroxyethyl methacrylate (HEMA) with a photoinitiator (for example, 1% by weight relative to the HEMA monomer) and can thus be printed tomographically. The photoinitiator is chosen such that it has good dissolution in the extraction liquid. The monomer, by contrast, is chosen such that it cannot be dissolved in the extraction liquid. After the tomographic printing, the printed intraocular lens is then introduced into the defined extraction liquid or the defined extraction liquid is introduced into the vessel containing the printed intraocular lens. The printed intraocular lens then remains in the defined extraction liquid for a defined period of time. Within this period of time, the soluble photoinitiator is dissolved in the defined extraction liquid, which penetrates into the at least one cavity through the (permeable) wall (for example, poly-HEMA) of the at least one cavity and washes the dissolved photoinitiator out of the at least one cavity. After the defined period of time, the proportion of the photoinitiator in the remaining liquid in the at least one cavity tends to zero and it is no longer possible to activate the curing or polymerization.

In an alternative embodiment, the curable liquid includes a monomer that is soluble with respect to the defined extraction liquid, where the monomer present in the at least one cavity is removed from the at least one cavity by the impregnating and/or extracting operation. In principle, the procedure here is analogous to the embodiment described above.

In particular, in one embodiment, the defined extraction liquid is water.

In one embodiment, the curable liquid includes a macromonomer having a large molar mass (in particular >1000 g/mol). The photoinitiator can thus be removed from the at least one cavity by the impregnating and/or extracting while the macromonomer remains in the cavity. If water is used as extraction liquid, the water-insoluble monomer used may be, for example, a polymethylmethacrylate (PMMA) macromonomer.

In one embodiment, the printed intraocular lens with the at least one cavity is exposed to a chain-terminating reagent in the deactivating operation. It is envisaged here in particular that the chain-terminating reagent diffuses through the polymerized outer wall of the at least one cavity into the liquid present in the at least one cavity and prevents further activation of the curable liquid therein. Examples of chain-terminating reagents are chemical substances including halogens or halogen radicals (for example, bromine). These can either add on to the terminal acrylate (for example, Br2) or even cause an intramolecular cyclization reaction, both of which lead to deactivating of the monomer.

In one embodiment, in particular, the chain-terminating reagent is or includes at least one of the following: oxygen or hydrogen. Oxygen and hydrogen can suppress free-radical chain polymerization.

In one embodiment, the printed intraocular lens exposed to the chain-terminating reagent in the deactivating operation is irradiated with electromagnetic radiation of a defined wavelength or a defined wavelength range. This can achieve the effect that the monomers remaining in the at least one cavity are activated. There is a high probability here that the remaining monomers will react with the chain-terminating reagent present, such that only oligomers with a small molar mass are formed, which are not crosslinked and therefore remain liquid. The example that follows illustrates the basic procedure: For example, the curable liquid may include HEMA with a photoinitiator that has an absorption maximum at a wave length of about 400 nm. HEMA has an absorption range down to a wavelength of about 250 nm or lower. In a first step, a light source with a wavelength in the region of 400 nm is used to polymerize the HEMA in the curable liquid by tomographic printing. After the tomographic printing, the HEMA-filled at least one cavity of the intraocular lens is exposed to an environment including the chain-terminating reagent (for example, oxygen, hydrogen, water). Gases can move unhindered into the monomer-filled structure. In the case of liquids, this depends on the chemical structure of the wall of the at least one cavity. Subsequently, the intraocular lens with the at least one cavity is irradiated with UV light with a wavelength of about 250 nm or lower. This triggers the activation of the remaining HEMA monomers having an acrylate group. As already described above, these monomers are highly likely to react with the chain-terminating reagent and only HEMA oligomers with low molar mass are produced, which are not crosslinked and therefore remain liquid.

In one embodiment, the curable liquid includes a crosslinker having two or more acrylate or methacrylate (end) groups for polymerization and a covalently bonded organic molecular chain in between that has at least one photocleavable group which is cleaved on irradiation with electromagnetic radiation in the UV wavelength range and leads to cleavage of the molecular chain, where a photoinitiator that is used in the curable liquid can be activated at a wavelength greater than the UV wavelength range, where the photoinitiator in the curable liquid is activated during the tomographic printing at a wavelength greater than the UV wavelength range, such that the molecular chain is not cleaved by the tomographic printing, where the printed intraocular lens with the at least one cavity, for cleavage of the photocleavable group, is irradiated with electromagnetic radiation in the UV wavelength range in the deactivating operation. Possible crosslinkers may be, for example: trimethylolpropane trimethacrylate, ethylene glycol dimethacrylate, butane-1,4-diol diacrylate, phenylene 1,4-diacrylate. But the method is not limited to these examples; it is also possible in principle to use other crosslinkers. The photocleavable group used may, for example, be phenacyl, which is an aromatic substituent with a phenyl attached to an acyl group. This phenacyl group is among those known for the fact that a photodeprotection reaction takes place on irradiation in the UV wavelength range, which leads to cleavage of the molecular chain between the crosslinking end groups. In order not to trigger this cleavage during the tomographic printing, the photoinitiator has to be chosen such that it can be activated at a greater wavelength, in particular in a wavelength range above the UV wavelength range, that is, especially in the visible wavelength range.

In one embodiment, the deactivating includes a thermal activation. In particular, this embodiment involves using a monomer that is photochemically cured and thermally deactivated in the process disclosed.

Further features relating to the configuration of the assembly will be apparent from the description of configurations of the method. The advantages of the assembly are the same in each case as in the configurations of the method.

### BRIEF DESCRIPTION OF DRAWINGS

The invention will now be described with reference to the drawings wherein:

FIG. 1 is a schematic diagram of embodiments of the method;

FIG. 2 is a schematic diagram for elucidation of one embodiment of the method;

FIG. 3 is a schematic diagram for elucidation of further embodiments of the method; and,

FIG. 4 is a schematic diagram of one embodiment of the assembly.

### DETAILED DESCRIPTION

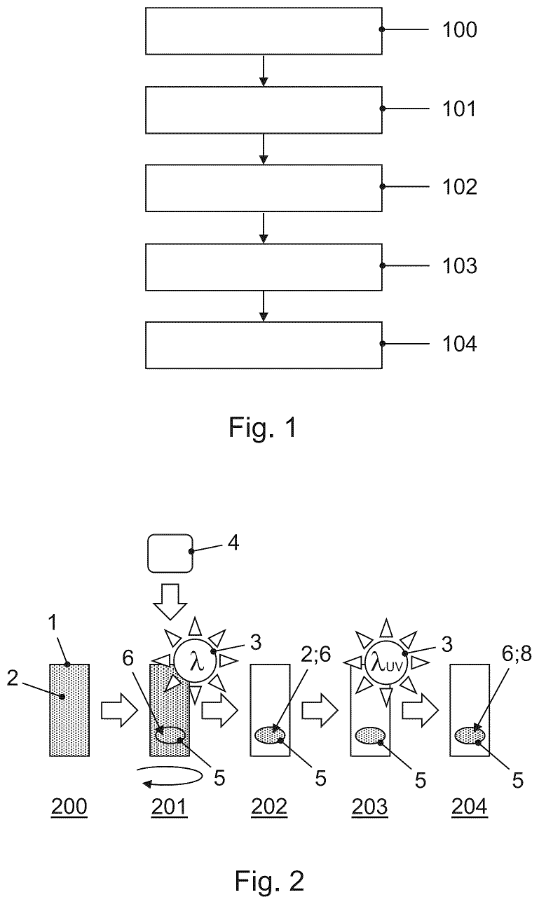

FIG. 1 shows a schematic diagram of embodiments of the method. In a method step 100, a vessel transparent to electromagnetic radiation is provided. The vessel is chosen here in particular such that it is transparent at least to the electromagnetic radiation used in the method, in particular in the visible and UV wavelength range.

In a method step 101, a liquid curable by electromagnetic radiation is provided in the vessel. In particular, this is a liquid containing monomers and a photoinitiator, where the monomers can be polymerized by activating the photoinitiator. The curable liquid is introduced, for example, into the vessel.

In a method step 102, a dataset of images of an intraocular lens with at least one cavity is created and/or provided, the images containing projections of the intraocular lens with the at least one cavity from different directions. The images are created in particular proceeding from three-dimensional data (for example, a CAD model of the intraocular lens with the at least one cavity) by calculating projections of electromagnetic radiation from different directions through the three-dimensional intraocular lens with the at least one cavity, which describe absorption of light by the material of the intraocular lens with the at least one cavity. This is an inverse process as used in tomographic imaging. For example, tomographic imaging is used in computed tomography. For example, tomographic imaging can make use of Radon transform or the reverse transform thereof.

In a method step 103, the curable liquid is tomographically printed via electromagnetic radiation proceeding from the dataset created and/or provided to form the intraocular lens with the at least one cavity. The curable liquid is irradiated here with electromagnetic radiation from different directions proceeding from the images of the dataset created and/or provided, as described, for example, in DE 10 2020 108 375 B3. In other words, the images are projected from a respectively corresponding direction into the curable liquid via electromagnetic radiation. As a result, the curable liquid is polymerized and cures at the respectively desired sites, thereby forming the intraocular lens with the at least one cavity.

In a method step 104, the curability of the curable liquid in the at least one cavity is deactivated. Further curing of the curable liquid in the at least one cavity is then no longer possible. In particular, the monomers present in the liquid remain in liquid form and can no longer be polymerized.

It may be the case in method step 104 that the deactivating includes impregnating and/or extracting the printed intraocular lens with and/or in a defined extraction liquid or a defined extraction gas. In particular, it is possible thereby to deactivate the monomer used and/or the photoinitiator used by impregnating in a solution (for example, a diene, and reaction of the (meth)acrylate via Diels-Alder reaction) or exposing to a suitable gas (for example, hydrogen/oxygen, deactivation of the acrylate function/immediate chain termination). The extracting can be effected, for example, with a hydrophobic extraction liquid (for example, hexane). This can remove photoinitiators, unpolymerized monomers and incompletely polymerized low molecular weight oligomers from the polymer and hence effectively end further polymerization.

This can be further developed in that the curable liquid includes a monomer that is insoluble with respect to the defined extraction liquid and a photoinitiator that is soluble with respect to the defined extraction liquid, where the photoinitiator present in the at least one cavity is removed from the at least one cavity by the impregnating and/or extracting operation. For example, it is possible to use a water-soluble photoinitiator (for example, potassium persulfate or 4,4′-azobis(4-cyanovaleric acid, ACVA)), which is removed from the cavity with unreacted monomer remaining therein. The monomer (for example, octadecyl methacrylate) is water-insoluble and therefore remains in the cavity. In this way, it is possible in particular to prevent curing of the remaining monomer in the cavity.

This can alternatively be further developed in that the curable liquid includes a monomer that is soluble with respect to the defined extraction liquid, where the monomer present in the at least one cavity is removed from the at least one cavity by the impregnating and/or extracting operation. For example, it may be the case that both the photoinitiator used (for example, ACVA) and the monomer used (for example, hydroxymethyl methacrylate) are water-soluble. The extraction liquid (water in the example) is used to remove both the photoinitiator and the monomer from the cavity and to fill up the cavity with the extraction liquid (water in the example). In principle, other solvents may also be used instead of water, in which case the photoinitiator and the monomer are selected accordingly.

In particular, it may be the case that the defined extraction liquid is water. In the first alternative, the monomer is then water-insoluble and the photoinitiator water-soluble. In the second alternative, the monomer is then water-soluble.

It may be the case that the curable liquid includes a macromonomer having a large molar mass (in particular >1000 g/mol). The macromonomer may, for example, be PMMA. In particular, the macromonomer is combined with a secondary monomer. In this regard, there is a multitude of possible combinations.

Table 1 below shows examples of macromonomers and secondary monomers, which are representative but not limiting. For example, one of the PMMA prepolymers can also be combined with one or more of the secondary monomers in the table. It is also possible to mix different prepolymers (for example, PMMA prepolymers of different lengths, or prepolymers of PMMA with prepolymers of the secondary monomers). Furthermore, a mixture component can be varied with any possible ratio to one another in order to obtain desired properties of the resultant copolymer.

TABLE 1
Examples of formulations of PMMA macromonomers and various
copolymer candidates. In this case, any of the macromonomers
(prepolymers) may be joined to any of the secondary monomers.
The proportions are also variable.

| PMMA | | |
|---|---|---|
| macromonomer | Secondary monomer | Crosslinker |
| (C₅O₂H₈)₅₀₋₁₀₀ | Hydroxyethyl methacrylate | Trimethylolpropane |
| (C₅O₂H₈)₁₀₀₋₂₀₀ | Ethoxyethyl methacrylate | trimethacrylate |
| (C₅O₂H₈)₅₀₀₋₇₀₀ | Ethylene glycol dimethacrylate | |
| (C₅O₂H₈)₈₀₀₋₁₀₀₀ | Butyl acrylate | |
| (C₅O₂H₈)₁₀₀₀₋₁₅₀₀ | Ethyl methacrylate | |

In method step 104, it may be the case that the printed intraocular lens with the at least one cavity is exposed to a chain-terminating reagent in the deactivating operation.

In particular, it may be the case that the chain-terminating reagent is or includes at least one of the following: oxygen, hydrogen, water.

It may also be the case that the chain-terminating reagent used is a chemical substance including halogens or halogen radicals (for example, bromine). These may then either add on to the terminal acrylates (for example, Br2) or even start an intramolecular cyclization reaction, both of which lead to deactivation of the monomer, such that no further curing or polymerization is possible thereafter.

It may also be the case in method step 104 that the printed intraocular lens exposed to the chain-terminating reagent in the deactivating operation is irradiated with electromagnetic radiation

n of a defined wavelength or a defined wavelength range. This results in activation of the monomers in the curable liquid; these react with the chain-terminating reagent (for example, oxygen, hydrogen or water) and only oligomers with a small molar mass are formed, which are not crosslinked and therefore remain liquid. The defined wavelength or the defined wavelength range is in particular in the UV range, that is, especially at wavelengths of 250 nm or lower.

It may be the case that the curable liquid includes a crosslinker having two or more acrylate or methacrylate (end) groups for polymerization and a covalently bonded organic molecular chain in between that has at least one photocleavable group which is cleaved on irradiation with electromagnetic radiation in the UV wavelength range and leads to cleavage of the molecular chain, where a photoinitiator that is used in the curable liquid can be activated at a wavelength greater than the UV wavelength range, where the photoinitiator in the curable liquid is activated during the tomographic printing in method step 103 at a wavelength greater than the UV wavelength range, such that the molecular chain is not cleaved by the tomographic printing, where the printed intraocular lens with the at least one cavity, for cleavage of the photocleavable group, is irradiated with electromagnetic radiation in method step 104 in the UV wavelength range in the deactivating operation.

In method step 104, it may be the case that the deactivating includes a thermal activation.

FIG. 2 shows a schematic diagram for elucidation of one embodiment of the method. The basic procedure is the same here as already described with reference to FIG. 1.

In a vessel 1 transparent to electromagnetic radiation, a liquid 2 curable by electromagnetic radiation is provided in a method step 200. The curable liquid 2 includes a crosslinker including two or more acrylate or methacrylate (end) groups for polymerization and a covalently bonded organic molecular chain in between that has at least one photocleavable group. The photocleavable group is cleaved on irradiation with electromagnetic radiation in the UV wavelength range, which leads to cleavage of the molecular chain. Also used in the curable liquid 2 is a photoinitiator that can be activated at a wavelength greater than the UV wavelength range.

In a method step 201, the curable liquid 2 is tomographically printed via electromagnetic radiation 3 proceeding from a dataset 4 created and/or provided to form an intraocular lens 5 with the at least one cavity 6. The dataset 4 includes images of the intraocular lens 5 with the at least one cavity 6, the images including projections of the intraocular lens 5 with the at least one cavity 6 from different directions. The dataset 4 can be calculated, for example, from a CAD model of the intraocular lens 5 with the at least one cavity 6 by calculating projections from different directions using the CAD model, which represent site- and material-dependent attenuation of the electromagnetic radiation. The basic procedure is described, for example, in DE 10 2020 108 375 B3.

In the tomographic printing in process step 201, the photoinitiator in the curable liquid 2 is activated at a wavelength λ greater than the UV wavelength range λ_{UV}, such that the covalently bound organic molecular chain is not cleaved by the tomographic printing.

After the tomographic printing, the curable liquid 2 is removed from the vessel 1 in a method step 202. The printed intraocular lens 5 remains in the vessel 1, with the at least one cavity 6 closed by an outer shell of the intraocular lens 5 and still containing the curable liquid 2.

In a method step 203, the printed intraocular lens 5 with the at least one cavity 6, for cleavage of the photocleavable group, in the course of deactivating, is irradiated with electromagnetic radiation 3 in the UV wavelength range λ_{UV}. This leads to cleavage of the molecular chain between the crosslinker end groups. In this way, it is possible to deactivate the liquid remaining in the at least one cavity 6, such that an intraocular lens 5 with a deactivated liquid 8 can be provided in a method step 204. Curing of the deactivated liquid 8 can then no longer be initiated via electromagnetic radiation 3, and so the liquid state is maintained.

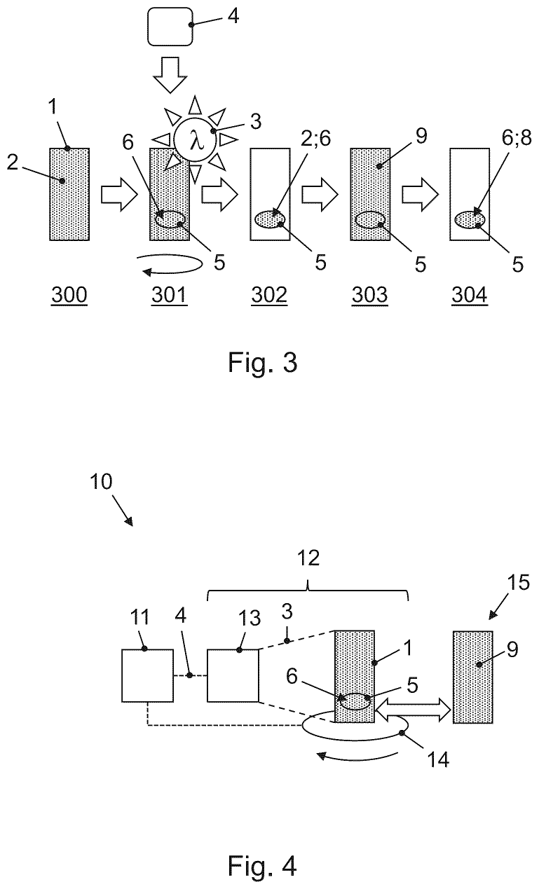

FIG. 3 shows a schematic diagram for elucidation of further embodiments of the method. The basic procedure is the same here as already described with reference to FIG. 1.

In a vessel 1 transparent to electromagnetic radiation, a liquid 2 curable by electromagnetic radiation is provided in a method step 300. It is envisaged that the curable liquid 2 includes a monomer insoluble with respect to a defined extraction liquid, for example a macromonomer with a large molar mass (for example, a PMMA macromonomer), and a photoinitiator soluble with respect to the defined extraction liquid. The proportion of the photoinitiator is, for example, 1% by weight of the monomer in the curable liquid 2. Examples are given in table 2 below.

TABLE 2
Examples of photoinitiators and possible extractants. Any of the initiators
can be extracted with any of the extractants mentioned.

| Photoinitiator | Extractant |
|---|---|
| 2,2-dimethoxy-1,2-diphenylethan-1-one | hexane |
| 2-hydroxy-2-methyl-1-phenylpropanone | cyclohexane |
| 1-hydroxy-cyclohexyl phenyl ketone | isopropanol |
| 1,2-diphenyl-2,2-dimethoxyethanone (Irgacure 651) | DMSO |
| (2,4,6-trimethylbenzoyl)phosphine oxide | ethanol, methanol |
| (Irgacure 819) | |
| benzophenone | benzene |
| isopropylthioxanthone | DMSO |

In a method step 301, the curable liquid 2 is tomographically printed via electromagnetic radiation 3 proceeding from a dataset 4 created and/or provided to form an intraocular lens 5 with the at least one cavity 6. The dataset 4 can be created and/or provided in the same way as already described for the working example shown in FIG. 2.

In a method step 302, the curable liquid 2 is removed from the vessel 1. The printed intraocular lens 5 remains in the vessel 1, with the at least one cavity 6 closed by an outer shell of the intraocular lens 5 and still containing the curable liquid 2.

In a method step 303, the defined extraction liquid 9 is introduced into the vessel 1. In this way, the printed intraocular lens 5 with the at least one cavity 6 is impregnated with the defined extraction liquid 9. The photoinitiator present in the at least one cavity 6 is removed from the at least one cavity 6 by impregnating (and/or extracting). In this way, it is possible to deactivate the liquid remaining in the at least one cavity 6, such that an intraocular lens 5 with a deactivated liquid 8 can be provided in a method step 304. Curing of the deactivated liquid 8 can then no longer be initiated via electromagnetic radiation 3, and so the liquid state is maintained.

If the defined extraction liquid 9 is water, for example, a water-insoluble monomer and a water-soluble photoinitiator are used.

In an alternative embodiment, the curable liquid 2 includes a monomer that is soluble with respect to the defined extraction liquid 9, where the monomer present in the at least one cavity 6 is removed from the at least one cavity 6 by the impregnating (and/or extracting) operation. In the above example, in which the defined extraction liquid 9 is water, a water-soluble monomer is used. For example, it is possible to use a hydroxyethyl methacrylate (HEMA) monomer. The HEMA monomer is water-soluble and the polymerized HEMA of the shell of the intraocular lens 5 is water-permeable, and so the HEMA monomer can be removed from the at least one cavity 6 by the impregnating (and/or extracting) operation.

FIG. 4 shows a schematic diagram of an embodiment of the assembly 10 for producing an intraocular lens 5. The assembly 10 includes a vessel 1 transparent to electromagnetic radiation 3, a data processing device 11 set up to create and/or provide a dataset 4 of images of an intraocular lens 5 with at least one cavity 6, the images including projections of the intraocular lens 5 with the at least one cavity 6 from different directions.

The assembly 10 further includes a tomographic printing device 12 set up for tomographic printing of a curable liquid 2 provided in the vessel 1 via electromagnetic radiation 3 proceeding from the dataset 4 created and/or provided to form the intraocular lens 5 with the at least one cavity 6. The tomographic printing device 12 especially includes an irradiation device 13 which projects the individual images through the transparent vessel 1 into the curable liquid 2 via the electromagnetic radiation 3, thereby bringing about curing in a site-dependent manner. The printing device 12 also includes, for example, a turntable 14 with which a direction of irradiation can be altered such that the curable liquid 2 can be irradiated from different directions (for example, with angles of incidence between 0 and 360° relative to an axis of rotation of the turntable 14). It is also possible to provide multiple irradiation devices 13, each of which emits electromagnetic radiation 3 according to the respective image of the dataset 4 from different directions simultaneously into the curable liquid 2.

The assembly 10 further includes at least one deactivating means 15 set up to deactivate the curability of the curable liquid 2 in the at least one cavity 6 or to prepare for and/or assist the deactivating operation. For example, it may be the case that the deactivating means 15 provided is a liquid reservoir which is set up to introduce a defined extraction liquid 9 into the vessel 1 and to remove it again therefrom (indicated merely schematically in FIG. 4) in order thereby to bring about the deactivating (with or without additional electromagnetic radiation).

It is understood that the foregoing description is that of the preferred embodiments of the invention and that various changes and modifications may be made thereto without departing from the spirit and scope of the invention as defined in the appended claims.

### LIST OF REFERENCE SYMBOLS

1 transparent vessel
2 curable liquid
3 electromagnetic radiation
4 dataset
intraocular lens
6 cavity
8 deactivated liquid
9 extraction liquid
assembly
11 data processing device
12 tomographic printing device
13 irradiation device
14 turntable
deactivating means
100-104 method steps
200-204 method steps
300-304 method steps
λ wavelength (>λ_{UV})
λ_{UV} wavelength (UV range)

1. A method of producing an intraocular lens, the method comprising:
providing a vessel transparent to electromagnetic radiation;
providing a liquid curable by electromagnetic radiation in the vessel;
at least one of creating and providing a dataset of images of the intraocular lens with at least one cavity, the images including projections of the intraocular lens with the at least one cavity from different directions;
tomographically printing the curable liquid via the electromagnetic radiation proceeding from the dataset at least one of created and provided to form the intraocular lens with the at least one cavity; and,
deactivating the curability of the curable liquid in the at least one cavity.

2. The method of claim 1, wherein said deactivating the curability includes at least one of impregnating and extracting the printed intraocular lens with and/or in a defined extraction liquid or a defined extraction gas.

3. The method of claim 2, wherein the curable liquid includes a monomer that is insoluble with respect to the defined extraction liquid and a photoinitiator that is soluble with respect to the defined extraction liquid, where the photoinitiator present in the at least one cavity is removed from the at least one cavity by said at least one of the impregnating and extracting operation.

4. The method of claim 2, wherein the curable liquid includes a monomer that is soluble with respect to the defined extraction liquid, where the monomer present in the at least one cavity is removed from the at least one cavity by said at least one of the impregnating and extracting operation.

5. The method of claim 2, wherein the defined extraction liquid is water.

6. The method of claim 1, wherein the curable liquid comprises a macromonomer having a large molar mass.

7. The method of claim 1, wherein the printed intraocular lens with the at least one cavity is exposed to a chain-terminating reagent in said deactivating the curability.

8. The method of claim 7, wherein, in said deactivating the curability, the printed intraocular lens exposed to the chain-terminating reagent is irradiated with electromagnetic radiation of a defined wavelength or a defined wavelength range.

9. The method of claim 1, wherein the curable liquid comprises a crosslinker having two or more acrylate or methacrylate (end) groups for polymerization and a covalently bonded organic molecular chain in between that has at least one photocleavable group which is cleaved on irradiation with electromagnetic radiation in the UV wavelength range and leads to cleavage of the molecular chain; a photoinitiator that is used in the curable liquid is configured to be activated at a wavelength greater than the UV wavelength range; the photoinitiator in the curable liquid is activated during the tomographic printing at a wavelength greater than the UV wavelength range, such that the molecular chain is not cleaved by the tomographic printing; and, said deactivating the curability includes irradiating the printed intraocular lens with the at least one cavity, for cleavage of the photocleavable group, with electromagnetic radiation in the UV wavelength range.

10. An assembly for producing an intraocular lens, the assembly comprising:
a vessel transparent to electromagnetic radiation;
a data processing device configured to at least one of create and provide a dataset of images of an intraocular lens with at least one cavity, the images including projections of the intraocular lens with the at least one cavity from different directions;
a tomographic printing device configured for tomographic printing of a curable liquid provided in said vessel via electromagnetic radiation proceeding from the dataset at least one of created and provided to form the intraocular lens with the at least one cavity; and,
at least one deactivator configured to deactivate a curability of the curable liquid in the at least one cavity or to prepare for or assist deactivating the curability.
